(19) United States
(12) Patent Application Publication
(10) Pub. No.: US 2022/0087716 A1
(43) Pub. Date: Mar. 24, 2022

(54) DELIVERY ASSISTANCE DEVICE FOR VAGINAL BREECH DELIVERY

(71) Applicant: Li-Cheng LU, Taoyuan City (TW)

(72) Inventor: Li-Cheng LU, Taoyuan City (TW)

(21) Appl. No.: 17/076,667

(22) Filed: Oct. 21, 2020

(30) Foreign Application Priority Data
Sep. 18, 2020 (TW) 109132271

(51) Int. Cl. A61B 17/42

(52) U.S. Cl. CPC A61B 17/42; A61B 2017/4216

(57) ABSTRACT

The disclosure provides a delivery assistance device for vaginal breech delivery. The delivery assistance device is suitable for dealing with a breech fetus delivering from a maternal and configured to be disposed on the fetal head for assisting the fetal head to delivered out of the maternal. The delivery assistance device includes a plurality of segment plates and at least one handle. The segment plates are assembled to become a curved plate configured to be disposed on the fetal head. The handle is disposed at a side of the curved plate and configured to move the curved plate to change a position of the fetal head when an external force is applied thereto, such that the fetus head can be released from the maternal pubis and delivered out of the maternal.

### CROSS-REFERENCE TO RELATED APPLICATIONS

This non-provisional application claims priority under 35 U.S.C. § 119(a) on Patent Application No(s). 109132271 filed in Taiwan, R.O.C. on Sep. 18, 2020, the entire contents of which are hereby incorporated by reference.

### TECHNICAL FIELD

The disclosure relates to a delivery assistance device, more particularly to a delivery assistance device that can assist vaginal breech delivery of fetal head.

### BACKGROUND

A breech birth is when a fetus is born bottom first instead of head first, and it must be managed carefully to avoid birth injury. There is one percentage of the breech fetuses may occur the interference between the fetus head and the maternal pubis, causing the fetal head entrapment during the vaginal breech delivery. In general, the fetal head entrapment is a very serious complication of breech delivery caused by the failure of the fetal head to negotiate the maternal pubis, and the fetal head entrapment will increase risk of brain damage due to hypoxia. One of the solution is to press the mother's lower abdomen to move the baby' head away from the maternal pubis. Another solution is to try to find the baby's face and then put pressure on the cheeks or on the mouth/chin in order to flex the head away from the maternal pubis. But these solutions highly rely on very experienced doctor and still have high failure possibility. Thus, in most of time, an invasive approach or a surgery is still necessary, increasing the risk to the maternal and the fetus.

### SUMMARY OF THE INVENTION

The disclosure provides a delivery assistance device that can effectively and safely deliver the fetal head during vaginal breech delivery.

One embodiment of the disclosure provides a delivery assistance device for vaginal breech delivery. The delivery assistance device is suitable for dealing with a breech fetus delivering from a maternal and configured to be disposed on the fetal head for assisting the fetal head to delivered out of the maternal. During the vaginal breech delivery, the fetus is born the bottom first instead of the head first, thus the fetus head may be interfered with the maternal pubis, causing the fetal head entrapment. The delivery assistance device includes a plurality of segment plates and at least one handle. The segment plates are assembled to become a curved plate configured to be disposed on the fetal head. The handle is disposed at a side of the curved plate and configured to move the curved plate to change a position of the fetal head when an external force is applied thereto, such that the fetus head can be released from the maternal pubis and delivered out of the maternal pelvis.

According to the delivery assistance device as discussed above, the curved plate being assembled by the segment plates can be pushed into the birth canal and placed under the fetal head, such that gently and properly pulling the handle can move the cheeks or the mouth/chin towards the chest via the curved plate in order to flex the fetal head to a position does not interfere with the maternal pubis. As a result, the fetal head is released from the maternal pubis. Thus, with the help of the delivery assistance device, the fetal head can be effectively and safely delivered during vaginal breech delivery.

### BRIEF DESCRIPTION OF THE DRAWINGS

The present disclosure will become better understood from the detailed description given herein below and the accompanying drawings which are given by way of illustration only and thus are not intending to limit the present disclosure and wherein:

FIG. 1 is a perspective view of a delivery assistance device for assisting vaginal breech delivery according to a first embodiment of the disclosure;

FIG. 2 is a partial exploded view of the delivery assistance device in FIG. 1;

FIG. 3 is a schematic view showing a vaginal breech delivery;

FIG. 4 is a schematic view showing that one segment plate of the delivery assistance device is pushed into the birth canal;

FIG. 5 is a schematic view showing that two segment plates of the delivery assistance device being pushed into the birth canal are assembled to each other;

FIG. 6 is a schematic view showing that a plurality of segment plates are assembled into a curved plate and placed on the fetal head;

FIG. 7 is a schematic view showing that the fetal head is moved by the curved plate;

FIG. 8 is a schematic view showing that the fetal head is moved out of the birth canal with the help of the curved plate;

FIG. 9 is a schematic view showing another way of using the curved plate of the delivery assistance device on the fetal head; and

FIG. 10 a schematic view showing that a delivery assistance device according to a second embodiment of the disclosure is applied on the fetal head.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In addition, the terms used in the present disclosure, such as technical and scientific terms, have its own meanings and can be comprehended by those skilled in the art, unless the terms are additionally defined in the present disclosure. That is, the terms used in the following paragraphs should be read on the meaning commonly used in the related fields and will not be overly explained, unless the terms have a specific meaning in the present disclosure.

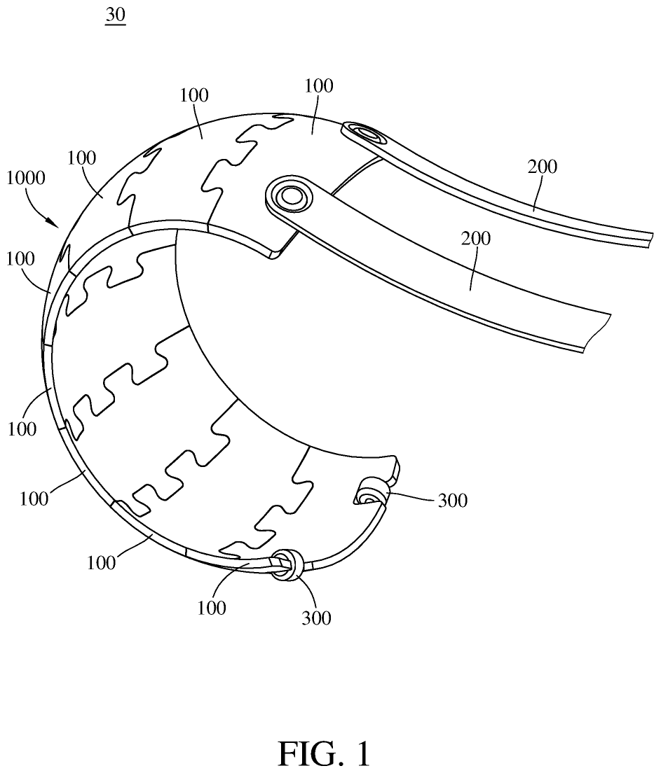

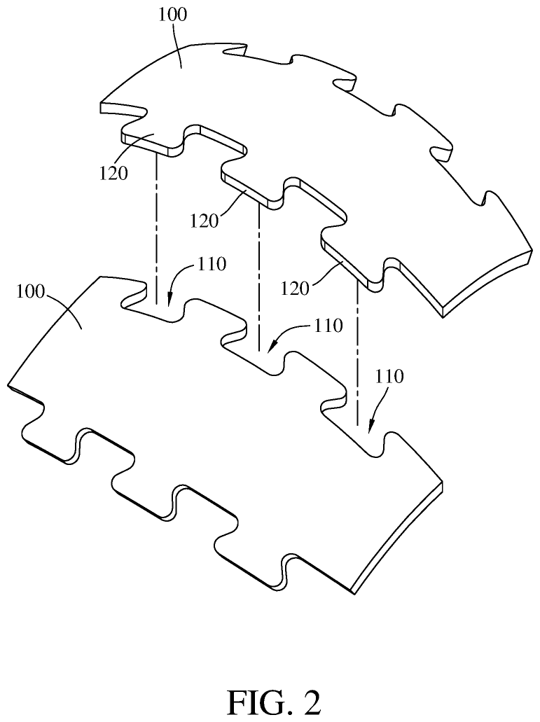

Referring to FIGS. 1 and 2, there are shown a perspective view of a delivery assistance device 30 for vaginal breech delivery according to a first embodiment of the disclosure and a partial exploded view of the delivery assistance device 30 in FIG. 1.

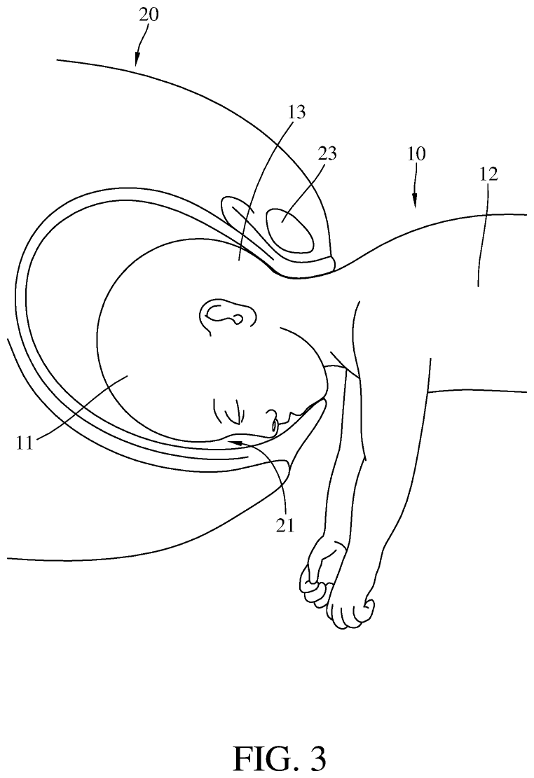

In this embodiment, the delivery assistance device 30 is configured to assist in delivering the fetal head during vaginal breech delivery. For example, as shown in FIG. 3, the delivery assistance device 30 is suitable for dealing with a fetus 10 in the breech presentation. The delivery assistance device 30 can be placed on or under the fetal head 11 when using. To achieve this, in this embodiment, the delivery assistance device 30 includes a plurality of segment plates 100 and two handles 200. In this or another embodiment, the delivery assistance device 30 may further include a plurality of rollers 300.

The segment plates 100 are made of, for example, acrylic, and each of them is in a curved shape. The segment plate 100 can be assembled into a curved plate 1000. In detail, the segment plates 100 can be assembled in series via, for example, dovetail-shaped protrusions 120 and mating recesses 110 therebetween. As shown, there are eight segment plates 100, and at least six of them each have a plurality of recesses 110 in dovetail shape and a plurality of protrusions 120 in dovetail shape, where the recesses 110 and the protrusions 120 are respectively formed at two opposite sides of the segment plate 100. The protrusions 120 can be inserted into the recesses 110 in tight contact manner, such that every two segment plates 100 can be stably fixed to each other.

Note that the protrusions and recesses of the segment plate are not restricted to be any particular shape as long as they can secure the connection between the segment plates. In some other embodiments, the protrusions and recesses of the segment plates may be in rectangular shape. On the other hand, the segment plates are not restricted to be assembled together via the protrusions and the recess. In some other embodiments, the segment plates may be assembled together via another structures, such as recesses and pins or latches.

The handles 200 are pivotably disposed on the same side of the curved plate 1000. As shown, the handles 200 may be symmetrically arranged with respect to the central line of the curved plate 1000. The rollers 300 are disposed on the same side of the curved plate 1000 that is opposite to the handles 200. That is, the rollers 300 and the handles 200 are respectively located two opposite ends of the curved plate 1000.

Then, referring to FIG. 3, there is shown a maternal 20 who presents in labor with a breech fetus 10. The fetus 10 is in the breech presentation, meaning that the fetus 10 is born bottom first instead head first, thus, in such a case, the fetal head 11 easily trapped at the maternal pubis 23 and therefore gets stuck in the birth canal 21 following delivery of the fetal body 12.

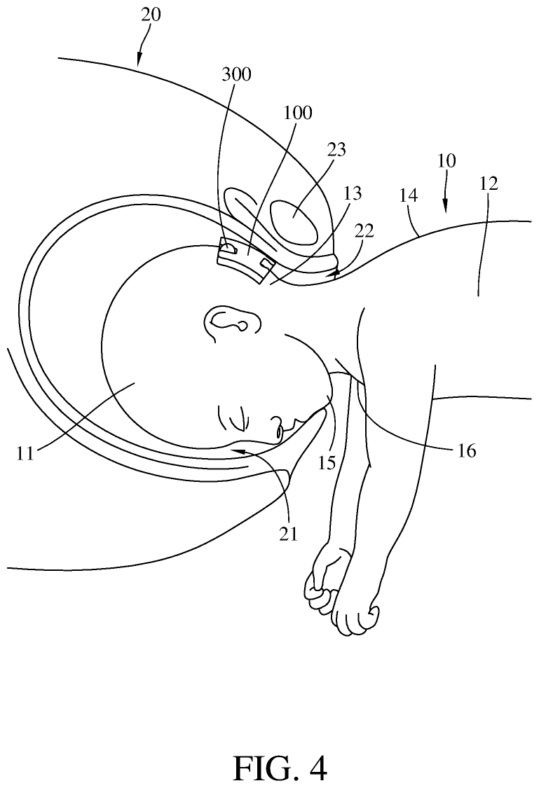

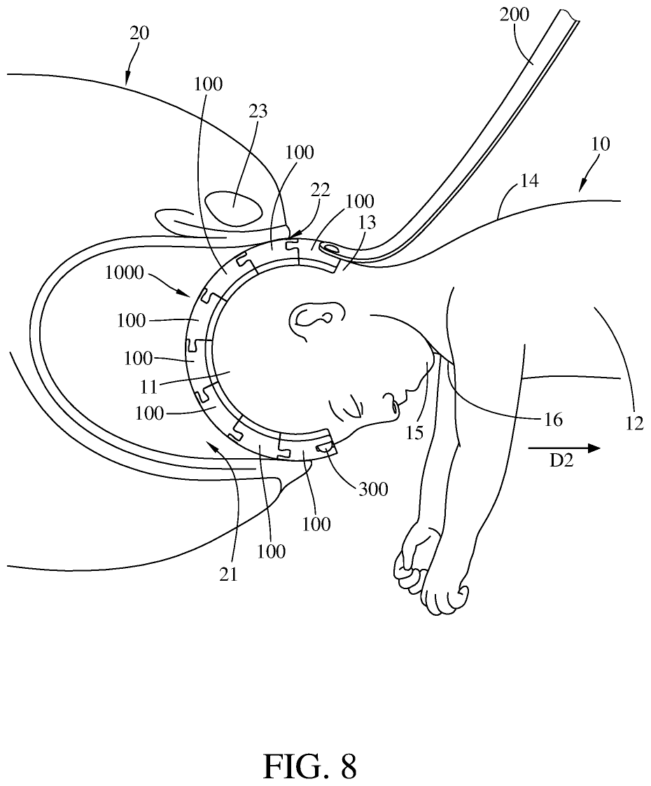

The following paragraphs will describe how to use the delivery assistance device 30 to free the fetus 10 from the birth canal 21. Referring to FIGS. 4 to 8, there are shown a schematic view showing that one segment plate 100 of the delivery assistance device 30 is pushed into the birth canal 21, a schematic view showing that two segment plates 100 of the delivery assistance device 30 being pushed into the birth canal 21 are assembled to each other, a schematic view showing that the segment plates 100 are assembled into a curved plate 1000 and placed on the fetal head 11, a schematic view showing that the fetal head 11 is moved by the curved plate 1000, and a schematic view showing that the fetal head 11 is moved out of the birth canal 21 with the help of the curved plate 1000.

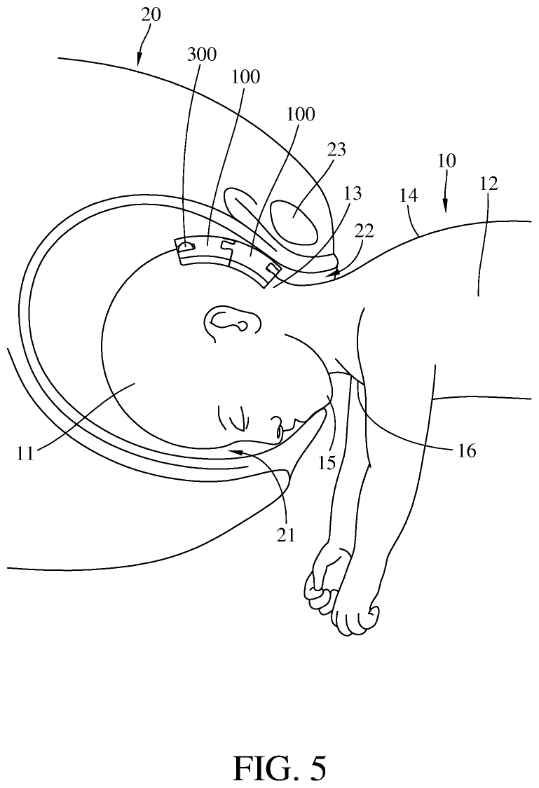

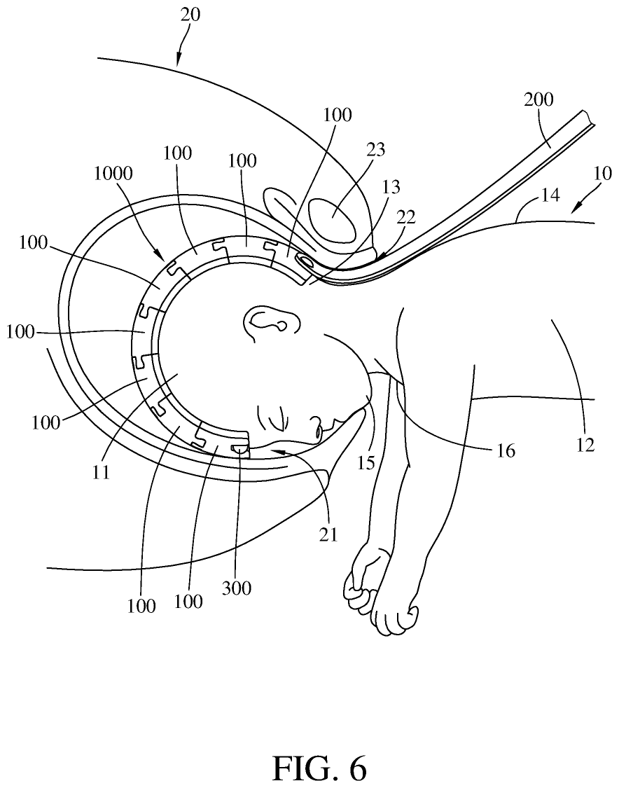

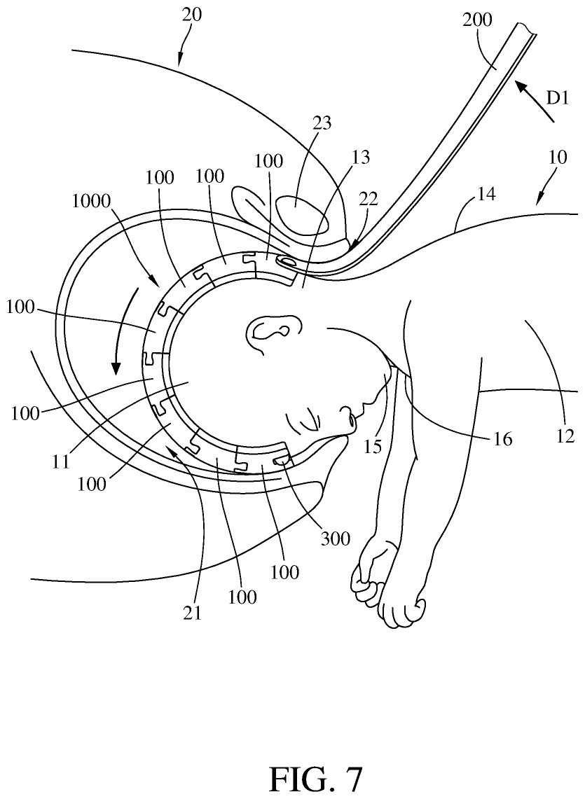

First step is to assemble the segment plates 100 in the birth canal 21. Specifically, as shown in FIG. 4, the segment plate 100 with the rollers 300 can firstly be placed into the birth canal 21 along the rear portion 13 of the fetal head 11, then, as shown in FIG. 5, another segment plate 100 is placed into the birth canal 21 following the same path and assembled with the segment plate 100 that has been in the birth canal 21. The push of the latter segment plate 100 can force the segment plate 100 with the rollers 300 to move further inwards the birth canal 21. The same step applies to the rest segment plates 100. And the segment plate 100 with the handles 200 is the last piece to be assembled. As a result, in FIG. 6, all of the segment plates 100 being pushed into the birth canal 21 are assembled to become the curved plate 1000. By doing so, the preparation is done, the curved plate 1000 covers on the fetal head 11, and the handles 200 remain outside the birth canal 21 and rest on the back 14 of the fetal body 12. Then, the handles 200 can be pulled to change the position of the fetal head 11 through the curved plate 1000. As shown in FIG. 7, properly and gently pulling the handles 200 (e.g., along the direction D1) can move the fetal head 11 to a position that the chin 15 is moved towards the chest 16 so that the rear portion 13 of the fetal head 11 is released from the maternal pubis 23 and finally can be delivered out of the birth canal 21. As such, the delivery assistance device 30 can provide a low-risk solution to assist the doctors for vaginal breech delivery.

Note that the rollers 300 can reduce the friction with the fetus 10 so as to facilitate the placement of the curved plate 1000 into the birth canal 21. However, the rollers 300 are optional in the disclosure; in some other embodiments, the delivery assistance device may not include any roller.

Note that the quantity of the handles 200 is not restricted to be two; in some other embodiments, the delivery assistance device may include more or less handle. In addition, in some other embodiments, the handle may be impivotable with respect to the curved plate.

Note that the quantity of the segment plates 100 is not restricted to be eight; in some other embodiments, the delivery assistance device may include more or less segment plates according to actual requirements.

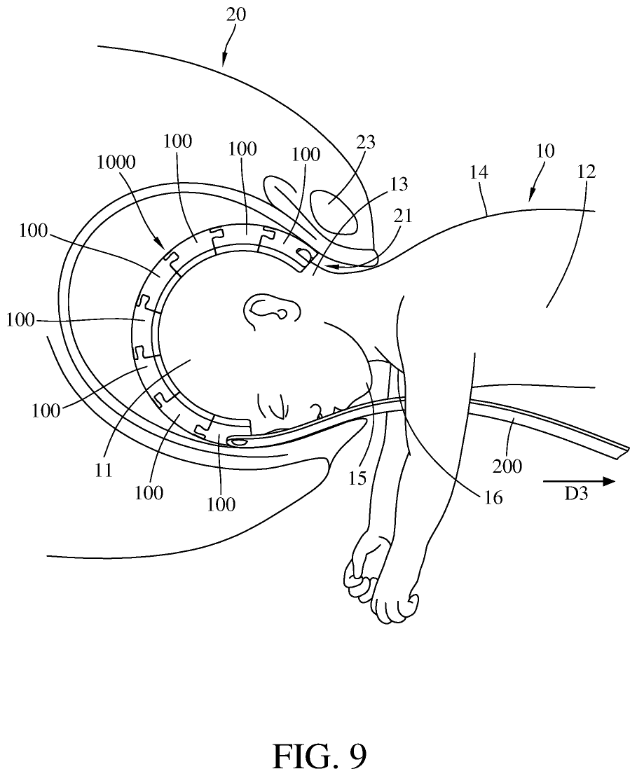

On the other hand, referring to FIG. 9, there is shown another schematic view showing another way of using the curved plate 1000 of the delivery assistance device 30 on the fetal head 11.

In this embodiment, the segment plates 100 can be placed into the birth canal 21 along the chin 15 of the fetus 10 and assembled with each other. When all of the segment plates 100 are pushed into the birth canal 21 and assembled to become the curved plate 1000, the curved plate 1000 covers on the fetal head 11 of the fetus 10, and the handles 200 are located in front of the chest 16 of the fetal body 12 of the fetus 10. At this moment, the handles 200 can be pulled (e.g., along a direction D3) to change the position of the fetal head 11 through the curved plate 1000, such that the rear portion 13 of the fetal head 11 can be released from the maternal pubis 23, thereby facilitating the delivery of the fetus 10.

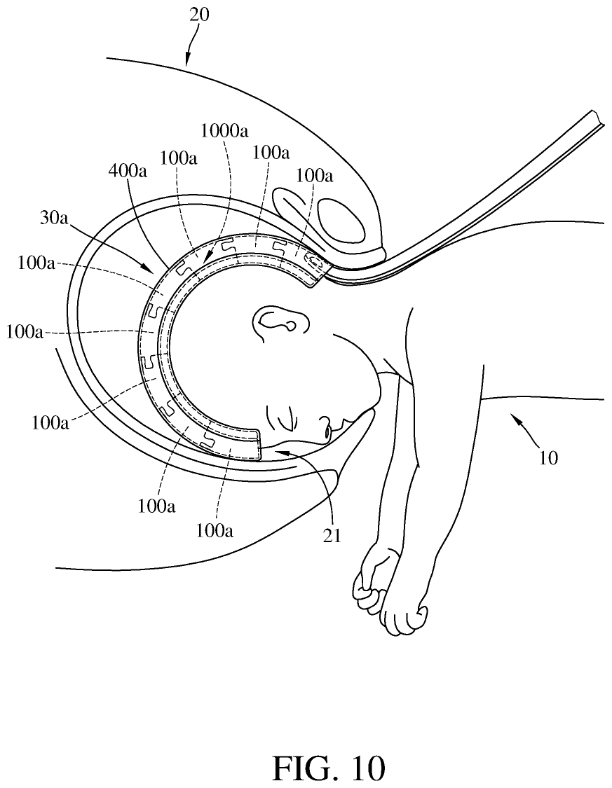

Then, referring to FIG. 10, there is shown a schematic view showing that a delivery assistance device 30a according to a second embodiment of the disclosure is applied on the fetal head 11.

In this embodiment, the delivery assistance device 30a is similar to the delivery assistance device 30 as shown in FIG. 1, therefore the following paragraphs will introduce the main difference between them, and the similar or same parts will not be further described hereinafter.

In this embodiment, the delivery assistance device 30a does not have any roller, but further includes a cloth cover 400a. The cloth cover 400a will be firstly sleeved on the first piece of the segment plates 100a before the first piece of the segment plates 100a is placed into the birth canal 21 of the maternal 20. After the cloth cover 400a is sleeved on the first piece of the segment plate 100a, the cloth cover 400a and the first piece of the segment plates 100a are together placed into the birth canal 21 of the maternal 20. Then, the second piece of the segment plates 100a are placed into the cloth cover 400a and assembled with the first piece of the segment plates 100a. The push of the second segment plate 100 can force the first piece of segment plate 100 to move further inwards the birth canal 21. The same step applies to the rest pieces of the segment plates 100. As shown in FIG. 10, after all of the segment plate 100a are assembled with each other and pushed into the birth canal 21, the cloth cover 400a is sleeved on a curved plate 1000a formed by the segment plates 100a, thereby preventing the segment plate 100a from scratching the fetus 10.

According to the delivery assistance device as discussed in the above embodiment of the disclosure, the curved plate being assembled by the segment plates can be pushed into the birth canal and placed under the fetal head, such that gently and properly pulling the handle can move the cheeks or the mouth/chin towards the chest via the curved plate in order to flex the fetal head to a position does not interfere with the maternal pubis. As a result, the fetal head is released from the maternal pubis. Thus, with the help of the delivery assistance device, the fetal head can be effectively and safely delivered during vaginal breech delivery.

It will be apparent to those skilled in the art that various modifications and variations can be made to the present disclosure. It is intended that the specification and examples be considered as exemplary embodiments only, with a scope of the disclosure being indicated by the following claims and their equivalents.

What is claimed is:

1. A delivery assistance device for vaginal breech delivery with a fetal head, comprising:
a plurality of segment plates, wherein the plurality of segment plates are assembled to become a curved plate configured to be disposed on the fetal head; and
at least one handle, disposed at a side of the curved plate and configured to move the curved plate to change a position of the fetal head when an external force is applied thereto.

2. The delivery assistance device according to claim 1, wherein the plurality of segment plates are assembled in series via protrusions and mating recesses therebetween.

3. The delivery assistance device according to claim 2, wherein the plurality of segment plates are assembled in series via dovetail-shaped protrusions and mating recesses therebetween.

4. The delivery assistance device according to claim 1, further comprising a plurality of rollers, wherein the plurality of rollers and the at least one handle are respectively disposed at two opposite of the curved plate.

5. The delivery assistance device according to claim 1, further comprising a cloth cover covering the curved plate.

6. The delivery assistance device according to claim 1, wherein each of the plurality of segment plates is made of acrylic.

7. The delivery assistance device according to claim 1, wherein the at least one handle comprises two handles, and the two handles are symmetrically disposed on the curved plate.

8. The delivery assistance device according to claim 1, wherein the at least one handle is pivotably disposed on the curved plate.

9. The delivery assistance device according to claim 1, wherein the plurality of segment plates comprises eight segment plates.
